SEQUENCE LISTING (16 sequences)

SEQ ID NO: 1 - DNA, 24 bp, Artificial Sequence
  PCR primer
ccttctggatcaacatggatgctg

SEQ ID NO: 2 - DNA, 26 bp, Artificial Sequence
  PCR primer
cgcctcttcctctaaatcgaagcagt

SEQ ID NO: 3 - DNA, 20 bp, Artificial Sequence
  PCR primer
atccctcgcagaccctatct

SEQ ID NO: 4 - DNA, 20 bp, Artificial Sequence
  PCR primer
taaactggggcaaggtgagt

SEQ ID NO: 5 - DNA, 20 bp, Artificial Sequence
  PCR primer
ggctgaaggactcactttgc

SEQ ID NO: 6 - DNA, 20 bp, Artificial Sequence
  PCR primer
tgaatcgactctccctcacc

SEQ ID NO: 7 - DNA, 20 bp, Artificial Sequence
  PCR primer
cgggatccattcaagagaca

SEQ ID NO: 8 - DNA, 25 bp, Artificial Sequence
  PCR primer
gctcgagccacacatccagtgcctt

SEQ ID NO: 9 - DNA, 20 bp, Artificial Sequence
  PCR primer
caaggtggctagaagacatc

SEQ ID NO: 10 - DNA, 23 bp, Artificial Sequence
  PCR primer
accactggaaacttattaacttc

SEQ ID NO: 11 - DNA, 20 bp, Artificial Sequence
  PCR primer
tgagaccttcaacaccccag

SEQ ID NO: 12 - DNA, 20 bp, Artificial Sequence
  PCR primer
catctgctggaaggtggaca

SEQ ID NO: 13 - DNA, 216 bp, Homo sapiens
  misc_feature: GLRA3_long
gtttcatatgtcaaagctattgatatttggatggcagtatgcctcctttttgtgttttcagcacttctggagtatgcagctgtaaattttgtatcaagacaacacaaagaacttctgagatttcgacgaaagagaaagaataagacagaagcttttgcactggagaagttttaccgtttctcagatatggatgatgaggtaagggaaagccgattc

SEQ ID NO: 14 - DNA, 171 bp, Homo sapiens
  misc_feature: GLRA3_short
gtttcatatgtcaaagctattgatatttggatggcagtatgcctcctttttgtgttttcagcacttctggagtatgcagctgtaaattttgtatcaagacaacacaaagaacttctgagatttcgacgaaagagaaagaataaggatgatgaggtaagggaaagccgattc

SEQ ID NO: 15 - DNA, 216 bp, Rattus norvegicus
  misc_feature: Glra3_rn_long
gtgtcctatgtcaaggcaattgacatttggatggcagtgtgtctcctttttgtgttctcagcacttctggagtatgcagccgtgaattttgtatcaaggcatcacaaagaactgctgaggtttcggcgaaagaggaaaaataaaacagaagcttttgcactggagaagttttaccgtttctcagacacggatgatgaggtgagggagagtcgattc

SEQ ID NO: 16 - DNA, 171 bp, Rattus norvegicus
  misc_feature: Glra3_rn_short
gtgtcctatgtcaaggcaattgacatttggatggcagtgtgtctcctttttgtgttctcagcacttctggagtatgcagccgtgaattttgtatcaaggcaacacaaagaactgctgaggtttcggcgaaagaggaaaaataaagatgatgaggtgagggagagtcgattc

(19) United States
(12) Patent Application Publication
(10) Pub. No.: US 2011/0077239 A1
(43) Pub. Date: Mar. 31, 2011

(54) GLYCINE RECEPTOR AGONISTS FOR THE TREATMENT OF PHANTOM PHENOMENA

(76) Inventors: Marlies Knipper, Leinfelden-Echterdingen Stetten (DE); Lukas Ruettiger, Kusterdingen (DE); Bernhard Schick, Hofbieber (DE); Julia Dlugaiczyk, Feucht (DE)

(21) Appl. No.: 12/818,391

(22) Filed: Jun. 18, 2010

Related U.S. Application Data
(63) Continuation of application No. PCT/EP2008/010759, filed on Dec. 17, 2008.

(30) Foreign Application Priority Data
Dec. 20, 2007 (DE) 10 2007 063 210.1

(51) Int. Cl. A61K 31/198; A61K 31/185; A61K 31/46; A61K 31/16; A61K 31/232; A61K 31/352; A61K 31/5383; A61K 31/5513; A61K 31/197; A61K 31/165; A61K 31/42; A61K 31/19; A61K 31/437; A61K 31/44; A61K 31/24; A61K 31/277; A61P 25/00; A61P 27/16; A61P 29/02

(52) U.S. Cl. 514/221; 514/561; 514/578; 514/304; 514/627; 514/549; 514/454; 514/230.2; 514/567; 514/619; 514/380; 514/302; 514/356; 514/534; 514/523

(57) ABSTRACT

A medicament for the treatment of the phantom phenomena of acute tinnitus and/or phantom pain, a method for the production of such a medicament, and a method for the treatment of such phantom phenomena

### CROSS-REFERENCES TO RELATED APPLICATIONS

This application is a continuation of copending International Patent Application PCT/EP2008/010759 filed on Dec. 17, 2008 and designating the United States, which was not published under PCT Article 21(2) in English, and claims priority of German Patent Application DE 10 2007 063 210.1 filed on Dec. 20, 2007, which is incorporated herein by reference.

### BACKGROUND OF THE INVENTION

1. Field of the Invention

The present invention relates to a medicament for the treatment of the phantom phenomena of acute tinnitus and/or phantom pain, a method for the production of such a medicament, and a method for the treatment of such phantom phenomena.

2. Related Prior Art

The phantom phenomenon of tinnitus refers to sounds perceived by a patient, which are generated by the ear or the auditory system. Tinnitus which only exists for a few weeks up to three months is designated as acute tinnitus. Tinnitus which exists for more than a year is designated as chronic tinnitus. According to epidemiologic surveys in Germany there are about three million adults. In all stages of life the numbers of those affected by chronic tinnitus vary between 4.4% and 15%. Globally seen each year about 10 million people develop tinnitus, which for about 340,000 people turn from the acute into a chronic form, so called incidence.

The causes of tinnitus are manifold and include chronic noise damages, acute explosion injuries of the auditory system, acute hearing loss, and other diseases associated with a hearing loss. Connections with an inner ear hearing loss in a chronically advancing form or in form of a noise induced hearing loss, followed by Morbus Meniàre and acute hearing loss are, according to clinical studies, for more than two-thirds connected to tinnitus. In addition, affections of the cervical spine and the temporomandibular joint and the masseter system are involved into the formation and maintenance of tinnitus. Tinnitus also seems to have a mental component, in that connection it is referred to psychogenic tinnitus. In many cases, however, despite an intensive diagnostics, a definite cause of tinnitus cannot be found.

At present the tinnitus therapy consists of psychosomatic treatment, relaxation therapy, biofeedback, hypnotherapy, electric stimulation, lidocaine, iontophoresis or masking. However, these are all exclusively symptomatic therapy concepts.

The WO 02/15907 A1 proposes the treatment of tinnitus by the administration of the potassium channel opener flupirtine. This treatment has the disadvantage that flupirtine is also a muscle relaxing analgesics, whereby an application is associated with non-tolerable side effects.

Wang et al. (2000), Evaluating effects of some medicine on tinnitus with animal behavioral model in rats, Zhonghua Er. Bi. Yan. Hou. Ke. Za. Zhi. 35 (5), abstract, suggest the administration of nimodipine for the treatment of tinnitus. Nimodipine is an inhibitor of the Ca⁺⁺ channel Cav1.3. However, it has turned out that the blockage of the Cav1.3 channel in the auditory system would immediately cause deafness so that nimodipine is absolutely inappropriate for the treatment of tinnitus.

The WO 2004/022069 A1 describes aberrant NMDA (N-methyl-D-aspartate) receptors as one of the potential causes of tinnitus. These altered so called glutamate receptor channels which are inter alia expressed by auditory nerve cells result in an increased influx of calcium into the cell. In this document it is suggested to use NMDA receptor antagonists for the treatment of tinnitus. It is absolutely unclear, however, whether with such substances the acute or chronic situation of the tinnitus is to be treated. Furthermore, no information is given how the substances are to be applied.

In the DE 101 24 953 A1a treatment concept for tinnitus is proposed consisting of the stimulation of the expression of the “brain-derived nerve growth factor” (BDNF). The authors of this document describe on the basis of an animal model that in chronic tinnitus the BDNF expression in the cochlea and in the colliculus inferior decreases for which reason the therapeutic approach as suggested by the authors consists in the stimulation of the BDNF expression. The authors of this document, however, have specifically and exclusively analyzed the situation in the chronic form of tinnitus. The rats used in this animal model were treated by salicylates over a time period of three months, thereby, as is known, inducing the chronic form of tinnitus; cf. Penner M. J. and Jastreboff P. J. (1996), Tinnitus: Psycho-physical observations in humans and animal models, in: Van de Water, Popper A. N., Fax, R. R. (Ed.), Clinical aspects of hearing, Springer, N.Y., Heidelberg, pages 208-304; and Bauer, C. A., et al. (1999), A behavioral model of chronic Tinnitus in rats. Otolaryngol. Head Neck Surg. 121, pages 457-462. However, it was not realized by the authors of DE 101 24 953 A1 that there have to be significant differences between the treatment of the chronic and the acute form of tinnitus.

In the WO 2006/079476 it is suggested that the phantom phenomenon of acute tinnitus and also of phantom pain could be treated by the use of a substance that interacts with the BDNF signal transduction cascade, such as e.g. a GABA receptor agonist.

An overview on the syndrome of tinnitus is given e.g. in Waddell, A., Canter, R. (2004), Tinnitus, Am. Fam. Physician 69, pages 591-592.

The phantom phenomenon of phantom pain refers to the projection of sensations into a part of the body, an extremity, the mamma, the rectum, a tooth and others, which have been amputated or denervated by a damage of the plexus or paraplegia. This part of the body is experienced as being present and after an amputation is felt like a swollen hand or foot, respectively, e.g. directly sitting on the stump.

Figures concerning the number of cases of amputations resulting in phantom pains are inconsistent and vary from 5 to 100%.

At present phantom pains are so far treated within the context of pain therapies, e.g. with anticonvulsants, baclofen or calcitonin. As a supportive measure sometimes pain distancing antidepressants are used. Also surgical methods are applied by means of which e.g. nerves can be blocked or stimulated. A targeted causal treatment method, however, does not exist so far, in particular because the molecular underlying mechanisms are not fully understood.

An overview on the syndrome of phantom pain can be found in Middleton, C. (2003), The causes and treatments of phantom limb pain, Nurs. Times 99, pages 30-33.

### SUMMARY OF THE INVENTION

It is, therefore, an objective underlying the invention to provide a new substance or a new concept of therapy, respectively, by means of which the phantom phenomena of acute tinnitus and phantom pain can be treated in a targeted manner, whereupon the disadvantages of the prior art should preferably be avoided.

This objective is achieved by the provision of a glycine receptor agonist.

It was surprisingly realized by the inventors that glycine receptors, which were so far predominantly detected in the central nervous system, are also expressed in the inner ear and can transmit inhibitory signals to the auditory nerve after a glycine receptor agonist has been bound. Furthermore, the inventors have realized that with the administration of a glycine receptor agonist and the resulting inhibitory signals the overactivity of the auditory nerve as observed in phantom phenomena can be corrected. Therefore, glycine receptor agonists are new substances by means of which phantom phenomena can be treated in a targeted manner.

The objective underlying the invention is, therefore, completely achieved by the provision of glycine receptor agonists.

It is preferred if as a glycine receptor agonist a substance is provided which is selected from the group consisting of D-alanine, L-alanine, L-serine, taurine, cannabinoid, tropine, nortropine and derivatives thereof.

This measure has the advantage that such agonistic substances are provided which highly affinely bind to the glycine receptor, which can be synthesized and formulated in a simple and cost effective manner. Preferred tropines and nortropine are described in Maksay et al. (2007), Synthesis of (nor)tropeine (di)esters and allosteric modulation of glycine receptor binding, Bioorg. Med. Chem., online publication of November 4; Maksay et al. (2007), Synthesis of tropeines and allosteric modulation of ionotropic glycine receptors, J. Med. Chem., 47(25): 6384-6391, disclose esters of 3 alpha- and 3 beta-hydroxy(nor)tropine and amids of 3 alpha aminotropines; Bíró T. and Maksay G. (2004), Allosteric modulation of glycine receptors is more efficacious for partial rather than full agonists, Neurochem. Int., 44(7): 521-527. The content of the before mentioned publication is incorporated herein by reference.

The cannabinoid that can be used as a glycine receptor agonist is preferably selected from the group consisting of anandamide, arachidonylglycerol, tetrahydrocannabinol, WIN 55,212-2.

This measure has the advantage that such cannabinoids are already provided which have been proven as being particularly suitable. Iatsenko et al. (2007), The synthetic cannabinoid analog WIN 55,212-2 potentiates the amplitudes of glycine-activated currents, Article in Ukrainian, Fiziol Zh., 53(3): 31-37 describe a cannabinoid having the designation of WIN 55,212-2, which represents a particularly efficient glycine receptor agonist. The content of the before mentioned publication is part of the present application.

According to a preferred further development of the use according to the invention the substance is administered locally at or into the ear, preferably via the round window membrane, or at the site of amputation.

This measure has the advantage that the substance is specifically administered to the site of action so that only small amounts of the active substance are required. As a result the organism of the patient is stressed to a lesser extent and side effects are largely reduced. In the case of the treatment of acute tinnitus the microdose system is ideal, which is described by Lehner, R. et al. (1996), A new implantable drug delivery system for local therapy of the middle and inner ear, Ear. Nose Throat 76, pages 567-570.

Alternatively, the local administration can be realized by the use of a biodegradable hydrogel serving as a carrier matrix for the glycine receptor agonist. Such a biodegradable hydrogel has been successfully used in an animal model for the local administration of BDNF to the round window of the inner ear; Ito et al. (2005), A new method for drug application to the inner ear, J. Otorhinolaryngol. Relat. Spec., pages 272-275.

Alternatively for the administration via the round window membrane gel pellets can be used, such as the products of Gelita®-Tampons, B. Braun, Melsungen AG, Germany. Alternatively, biocompatible nanoparticles can be taken into account such as e.g. described in Durán, J. D. et al. (2007), Magnetic colloids as drug vehicles, J. Pharm. Sci, online publication, Mohamed F. and van der Walle C. F. (2008), Engineering biodegradable polyester particles with specific drug targeting and drug release properties, J. Pharm. Sci, 97(1): 71-87, Abstract of December 2007; Gupta S, and Moulik S. P. (2008), Biocompatible microemulsions and their prospective uses in drug delivery, J. Pharm. Sci., 97(1): 22-45, Abstract published online in December 2007.

According to a further development of the invention the medicine comprises an additional substance which is active against phantom phenomena, which additional substance is selected from the group consisting of GABA receptor agonists, in particular benzodiazepines and substances related thereto, baclofen, gamma vinyl GABA, gamma acetylene GABA, progabid, muscimol, iboten, sodium valproate and tetrahydroisoxazolopyrdine (THIP), MAP kinase inhibitors, in particular U 0126 or PD 98058, Cam kinase inhibitors, L-type Ca⁺⁺ channel antagonists, in particular nicardipin or nifedipin or isradipin, CREP antagonists, glutamate antagonists, trkB antagonists.

This measure has the advantage that a medicine is provided which is particularly powerful against phantom phenomena. It is specifically taken advantage of the findings of the authors of WO 2006/079476, who have described that the before identified substances are suitable for a causal treatment of phantom phenomena.

A further subject matter of the present invention relates to a method for the production of a medicine for the treatment of the phantom phenomena of the acute tinnitus and/or the phantom pain in a human or animal being, comprising the following steps: (a) providing a glycine receptor agonist, and (b) formulating the glycine receptor agonist into a pharmaceutically acceptable carrier.

Pharmaceutically acceptable carriers and pharmaceutical adjuvants are well known in the prior art, cf. e.g. Kibbe A. H. (2000), Handbook of Pharmaceutical Excipients, 3^{rd} Edition, American Pharmaceutical Association and Pharmaceutical Press, the content of this publication is incorporated herein by reference.

It is to be understood that the before mentioned features and those to be explained in the following cannot only be used in the combinations as identified in each case but also in other combinations or in isolated form, without departing from the scope of the present invention.

The invention is now explained in detail by means of embodiments which are purely illustrative and which result in further features and advantages of the invention.

### BRIEF DESCRIPTION OF THE DRAWINGS

FIG. 1 detection GlyRα3, GlyRβ and gephyrin in the rat cochlea. The cDNA from the rat cochleae was analyzed by RT-PCR at different postnatal stages (P14, >P21). P14 and >P21, amplification of transcripts of GlyRα3 (512<bp), GlyRβ (732 bp), and gephyrin (573 bp). It is to be noticed that a double band can be observed for GlyRα3 (467 bp/512 bp, indicated by arrows) in adult animals. Transcripts of GlyRα1 and GlyRα2 were not detected. sc P21, spinal cord from P21 animals was used as positive control for GlyRα1-3, GlyRβ, gephyrin and β-actin. β-actin (655 bp) was used as a housekeeping gene;

FIG. 2 detection of GlyRα3_K and GlyRα3_L splice variants in the adult rat cochlea (>P21) a) GlyRα3 transcripts from the adult rat cochlea were amplified by RT-PCR. The double band is to be noted (indicated by double arrows). b) GlyRα3_K (467 bp) and GlyRα3_L (512 bp) splice variants were detected after the cloning of PCR fragments into the PCR-II-TOPO vector by means of Insert-PCR. c) Alignments of long and short cDNA sequences (Exon 8-10) of human GlyRα3 [GLRA3_L (SEQ ID No. 13), GLRA_K (SEQ ID No. 14)] and rat GlyRα3 [Glra3_rn-L (SEQ ID No. 15), Glra3_rn-K (SEQ ID No. 16)]. Identical nucleotides in all four sequences are marked with an asterisk. The 45 bp stretch of Exon 9 (in bold italics) is missing in the cDNA sequences of GLRA_K and Glra3_rn_K;

FIG. 3 mRNA expression of GlyRα, GlyRβ and gephyrin in the neurons of the spiral ganglions of the cochlea of the adult rat (>P21). a), b) expression of mRNA of GlyRα3 in neurons of the spiral ganglions (SG, arrow) at different magnifications by using the whole-mount in situ hybridization of the cochlea of the adult rat (>P21). The overview (a) displays also a labeling of the outer hair cells (OHC). c), d) the expression of the mRNA of GlyRβ in neurons of the spiral ganglions (SG, arrow). e), f) expression of the mRNA of gephyrin in neurons of the spiral ganglions (SG, arrow). Higher magnifications show the cytoplasmic staining of the mRNA of GlyRα3 (b), the mRNA of GlyRβ (d), and the mRNA of gephyrin (f) in SG. No signals were detected in the hybridization with the corresponding sense molecules (insets a to f). Scale bars in a), c), e) 200 μm, in b), d), f) 20 μm;

FIG. 4 The expression of the mRNA of GlyRα3, GlyRβ and gephyrin in the adult organ of Corti (>P21). a) GlyRα3 transcripts were detected in the outer hair cells (OHC, filled arrows) by whole-mount in situ hybridization. No signal was obtained for the inner hair cells (IHC, open arrows). b) OHCs (filled arrows), however not IHCs (open arrows) revealed a signal for GlyRβ transcripts in the cochleae of the adult rat. c) In IHC (open arrows) and OHCs (filled arrows) an intense hybridization signal was detected for gephyrin. No signals were detected upon hybridization with the corresponding sense molecules (insets). Scale bars 20 μm;

FIG. 5 GlyRα/GlyRα3 protein expression on the level of the IHC. a), b) By using the monoclonal antibody mAb4a which detects all of the GlyRα subunits, the GlyRα protein was detected in cryosections of the rat cochleae at P8 under the IHC (open arrowheads), shown for the apical and also for mid-basal cochlear turn. At this age no GlyRα protein was detected on the level of the OHCs. c) to e) By using the polyclonal GlyRα3 specific antibody on the level of the IHCs (*) GlyRα3 protein was detected in the whole-mount in situ hybridization of the cochleae of the adult rat [>P21) (c) e)]. A co-immuno labeling was performed by using an antibody against the neurofilament 200, a marker for afferent nerve fibers (NG 200, filled arrowheads). The specimens were counterstained with DAPI, highlighting the cell nuclei. Scale bars in a), b) 20 μm, in c) to e) 50 μm;

FIG. 6 GlyRα3 protein expression on the level of the OHCs in the adult organ of Corti (>P21). a) to c) GlyRα3 protein (*) was detected in OHCs (filled arrows) by whole-mount immunohistochemistry. The specimens were co-immunolabeled with anti-neurofilament 200 (NF 200, filled arrowheads). NF 200 stained the nerve fibers which terminate at the OHCs. The cell nuclei were counterstained with DAPI. d) to f) Higher magnifications of a) to c). It is noticeable that the GlyRα3 protein (*) is localized in the cell membrane of the OHCs (filled arrows) opposite to the nerve fiber terminals (filled arrowheads). Scale bars in a) to c) 50 μm, in d) to f) 20 μm;

FIG. 7 Measurements of compound action potentials (CAP) of the auditory nerve after local administration (LA) of strychnine (glycine receptor inhibitor). At high degrees of loudness (to the left) the CAP amplitudes further increase monotonously after the administration of strychnine, since the inhibition of the neurons of the auditory nerves which usually starts at high degrees of loudness fails to appear. This results in an over-stimulation at high degrees of loudness (FIG. 7a). CAP measurements after the local administration of taurine (glycine receptor agonist). At low to medium degrees of loudness (right) after the administration of taurine the CAP amplitudes increase slower, since the glycinergic (inhibitory) neurons which are usually inactive at low degrees of loudness, were activated by taurine and inhibit the auditory nerve. This results in an inhibition at low and medium degrees of loudness. At high degrees of loudness the glycinergic neurons are activated anyway, therefore an overlapping of the CAP amplitude function can at least be found over a limited range of loudness (FIG. 7b).

FIG. 8 Schematic diagram of the postulated glycinergic innervation of the inner (IHC) and outer hair cells (OHC) after the onset of hearing. The afferent dendrites (AF) of the neurons of the spiral ganglions (SG) below the IHCs are contacted by efferent fibers of the lateral cochlear bundle (EF-LOC), which forms axodendritic synapses. GlyRα3 (dots) is transported from the SG to the afferent dendrites below the IHCs, where the protein was detected (cf. FIG. 5). GlyRα3 protein was detected in the OHCs (cf. FIG. 6) which form axosomatic synapses with the afferent fibers of the medial oliviocochlear (MOC) bundle (ES-MOC);

FIG. 9 Schematic diagram of the increased expression of BDNF and the decreased expression of Arg3.1/Arc in the periphery of the cochlea as observed in tinnitus (a), and the correction by glycine receptor agonists (“glycine”) or GABA receptor agonists (“GABA”) (b).

### BRIEF DESCRIPTION OF PREFERRED EMBODIMENTS

### 1. Material and Methods

### 1.1 Animals

In the experiments adult female Wistar rats (Charles River, Sulzfeld, Germany) in the age of 2 to 4 months were used. The care and use of the animals and the experimental protocol were reviewed and approved by the Animal Welfare Commissioner and the Regional Board for Scientific Animal Experiments in Tüibingen.

### 1.2 Tissue Preparation

For RNA isolation, the cochleae were dissected and immediately frozen in liquid nitrogen. For in situ hybridization and the immunohistochemistry, the cochleae were isolated and prepared as previously described; cf. Knipper et al. (2000), Thyroid hormone deficiency before the onset of hearing causes irreversible damage to peripheral and central auditory systems, J. Neurophysiol. 83:3101-3112. Briefly, cochleae were fixed by injection of 2% paraformaldehyd/2% sucrose (all chemicals from SIGMA-Aldrich, Munich, Germany, unless indicated otherwise) in 50 mM phosphate-buffered saline (pH 7.4) into the round and oval window. Cochleae of the animals older than P10 were decalcified for 10 minutes to 2 hours in RBD (“rapid bone decalcifier”, Eurobio, Les Ulis Cedex, France) after fixation. After the injection of 25% of sucrose and 1 mM protease inhibitor (Pefabloc; Roche Diagnostics, Mannheim, Germany) in HEPES-Hanks solution, cochleae were embedded in O.C.T. compound (Miles Laboratories, Elkhart, Ind., USA), cryosectioned at 10 mM, mounted on SuperFrost*/plus microscope slides, dried for one hour, and stored at −20° C. before use. For each experiment at least three animals of the indicated age were used (n=3).

### 1.3 RT-PCR

Total RNA was extracted from rat cochleae with the RNeasy Mini Kit (Qiagen, Hilden, Germany). Reverse transcription (RT) into cDNA was performed using the “Sensiscript Reverse Transcription Kit” (Qiagen, Hilden, Germany) and oligo-dT₁₅ primers (Roche, Penzberg, Germany) following the manufacturer's instructions. For the polymerase chain reaction (PCR), the following primers were used (the size of the PCR product is given in brackets). Glra1, forward: 5′ CCTTCTGGATCAACATGGATGCTG 3′ (SEQ ID No. 1), reverse: 5′ CGCCTCTTCCTCTAAATCGAAGCAGT 3′ (SEQ ID No. 2), 243 bp); Glra2, forward: 5′ ATCCCTCGCAGACCCTATCT 3′ (SEQ ID No. 3), reverse: 5′ TAAACTGGGGCAAGGTGAGT 3′ (SEQ ID No. 4), (553 bp); Glra3, forward: 5′ GGCTGAAGGACTCACTTTGC 3′ (SEQ ID No. 5), reverse; 5′ TGAATCGACTCTCCCTCACC 3′ (SEQ ID No. 6) (Glra3 primers were designed to detect possible splice variants corresponding to the human GLRA3 splice variants α3_L and α3_K; α3_L: 513 bp, α3_K: 468 bp); Glrb, forward: 5′ CGGGATCCATTCAAGAGACA 3′ (SEQ ID No. 7), reverse: 5′ GCTCGAGCCACACATCCAGTGCCTT 3′ (SEQ ID No. 8) (732 bp); gephyrin, forward: 5′ CAAGGTGGCTAGAAGACATC 3′ (SEQ ID No. 9), reverse: 5′ ACCACTGGAAACTTATTAACTTC 3′ (SEQ ID No. 10) (573 bp); β-actin, forward: 5′ TGAGACCTTCAACACCCCAG 3′ (SEQ ID No. 11), reverse: 5′ CATCTGCTGGAAGGTGGACA 3′ (SEQ ID No. 12) (655 bp). Distilled water served as negative control.

The PCR was performed with PuReTaq Ready-To-Go PCR beads (Amersham Biosciences, Freiburg, Germany). The PCR program consisted of an initial denaturation phase of 3 min at 94° C., 35-40 cycles of denaturation at 94° C. (30 sec), annealing at 58° C. (30 sec), extension at 72° C. (90 sec) and a final synthesis step of 10 min at 72° C. The resulting PCR products were separated on agarose gels and stained with ethidium bromide. The PCR products of GlyRα3, GlyRβ and gephyrin were sequenced and compared to the corresponding sequence data from GeneBank by BLAST (www.ncbi.nlm.nih.gov). The designations of GlyRα3 Exons refer to the Ensemble automatic gene annotation system (www.ensembl.org) and are distinct from the original description by Nikolic et al. (1998), The human glycine receptor subunit alpha3. Glra3 gene structure, chromosomal localization, and functional characterization of alternative transcripts, J. Biol. Chem. 273:19708-19714.

### 1.4 Riboprobe Synthesis and In Situ Hybridization

For the specific riboprobes, the PCR fragments of GlyRα3_L (513 bp), GlyRβ (732 bp) and gephyrin (573 bp) were cloned into the pCR-II-Topo vector (Invitrogen, Karlsruhe, Germany) respectively and used for the in vitro transcription. The complementary strands for the sense and antisense probes were transcribed from either SP6 or T7 promotor sites in the presence of digoxigenin-labeling mix (DIG; Roche Diagnostics).

Whole-mount in situ hybridizations with GlyRα3L, GlyRβ and gephyrin riboprobes were performed as described; cf. Engel et al. (2006), Two classes of outer hair cells along the tonotopic axis of the cochlea, Neuroscience 143:837:849. Briefly, cochleae of rats of the indicated age were fixed with 2% paraformaldehyd for 30 min followed by dehydration in 100% methanol overnight at −20° C. After rehydration and digestion with proteinase K (2 μg/ml) at 37° C. for 3 min, cochleae were post-fixed in 2% paraformaldehyd for 15 min. DIG-labeled antisense or sense probes were diluted in hybridization solution containing 25 microarray hybridization buffer (Amersham Biosciences, Freiburg, Germany), 25% nuclease-free water and 50% formamid. The hybridization was carried out at 55° C. over night. The subsequent washing and detection steps were performed as described; cf. Knipper et al. (1999), Distinct thyroid hormone-dependent expression of TrKB and p75NGFR in nonneuronal cells during the critical TH-dependent period of the cochlea, J. Neurobiol. 38:338-356; Knipper et al. (2000, l.c.). Each hybridization was done in at least three different animals at a given age.

### 1.5 Fluorescence Immunohistochemistry

For the immunohistochemistry, rat cochlea sections were stained and imaged as described; cf. Knipper et al. (2000 l.c.) and Knipper et al. (1998), Thyroid hormone affects Schwann cell and oligodendrocyte gene expression at the glial transition zone of the VIIIth nerve prior to cochlea function, Development 125:3709-3718. The mouse monoclonal antibody mAb4a which is directed against a common N-terminal epitope of the GlyRα1-4 subunits, was obtained as hybridoma supernatant. For the specific detection of the GlyRα3 subunit, a rat polyclonal antibody (SIGMA-Aldrich) was used. Mouse monoclonal antibodies against gephyrin (BD Transduction Laboratories, Heidelberg, Germany) and neurofilament 200 (NF200; The Binding Site, Heidelberg, Germany) were used. Primary antisera were visualized with Cy3-(Jackson ImmunoResearch Laboratories, West Grove, Pa., USA) or Alexa488-conjugated secondary antibodies (Molecular Probes, Leiden, The Netherlands). Sections were mounted in Vectashield mounting medium containing DAPI nuclear staining composition (Vector Laboratories, Burlingame, Calif., USA). The specimens were photographed using an Olympus AX70 microscope equipped with epifluorescence illumination and 40× (numerical aperture 1.0) or 100× oil immersion objectives (numerical aperture 1.35). The images were acquired using a CCD color view 12 camera and imaging system analysis (SIS, Münster, Germany). Each staining was performed at least in triplicate in three animals of a given age and genotype. The immunohistochemical analyses were performed on postnatal day 8 (P8) or in the adult (>P21) rat cochleae. For the whole-mount immunohistochemistry the preparation and fixation of cochleae was performed as described in Engel et al. (2006, l.c.), and the immunohistochemistry protocols were followed as reported above.

### 1.6 Measurements of Compound Action Potentials (CAP) of the Auditory Nerve

To get an access to the inner ear (cochlea) the middle ear is opened by surgery: A small opening behind the ear drum enables the access to the round window of the cochlea, in the massive bone of the bony labyrinth this is the only non-traumatic access to the inner ear. A silver wire electrode having a surface melted silver pearl is carefully positioned through the opening on the membrane of the round window, secured with tissue glue and the opening is closed with dental cement. The silver wire is conducted in the neck of the animal through the skin towards the outside and can then directly be connected to the electrophysiology amplifier for the measurements. The direct electrical access through the CAP electrode enables the direct largely interference-free down lead of the compound potentials of the auditory nerves resulting from an acoustic stimulation. In the signal response, besides the compound action potentials of the auditory nerves (CAP), also the formation of the excitation in the outer hair sense cells (OHC) can be detected as cochlea microphone potentials (CM). These three potentials in the measurements in vivo provide information on: 1) the activity of the auditory nerve close to the threshold up to high levels of acoustic pressure and, therefore, also on the modulation of the transmission, which is mediated by a glycinergic efferent feedback to the afferences of the IHCs, 2) the integrity of the IHCs (via the course of the SPs at increasing levels of acoustic pressures), 3) the amplification by the OHC (through the phase synchronicity of the CM signals with the auditory stimulus).

### 1.7 Local Administration of Glycine Receptor Agonists and Antagonists

Via the retro-auricular access which was already prepared for the CAP electrode after a first control measurement a gelatine carrier substance (Geleter, Braun) is carefully introduced into the round window niche and soaked with 5 to 10 μl of the glycine receptor agonist taurine 10 mM f.c. and the glycine receptor antagonist strychnine 50 mM f.c.

### 2. Results

### 2.1 Amplification of Glycine Receptors and the Anchor Protein Gephyrin in the Mammalian Cochlea

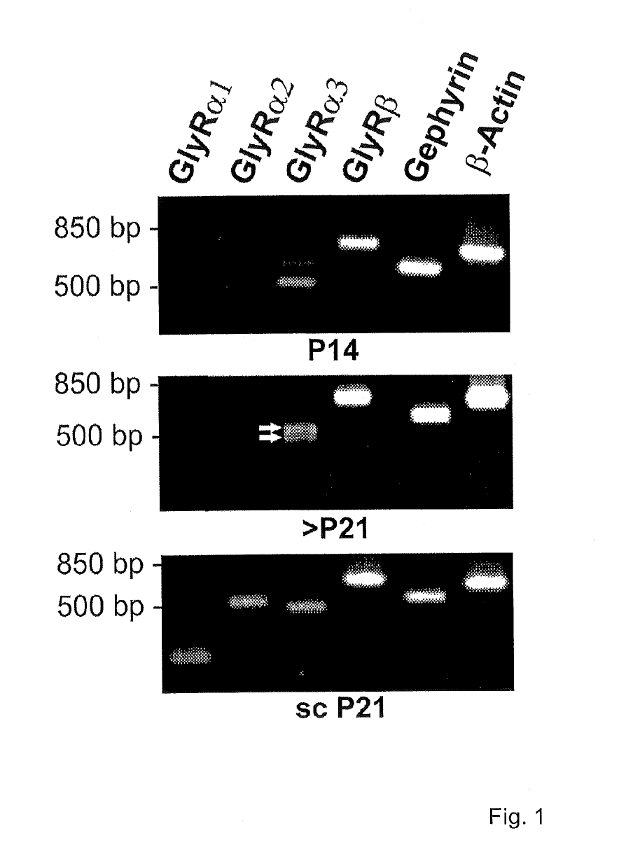

Using RT-PCR, GlyRα3 (512 bp), GlyRβ (732 bp) and gephyrin (573 bp) transcripts were amplified from rat cochlea at P14 (FIG. 1, P14). β-actin (655 bp) was used as a housekeeping gene. GlyRα3 primers were designed to detect possible splice variants corresponding to the human GLRA3 short (GlyRα3_K) and GLRA3 long (GlyRα3_L) subunit isoforms. RT-PCR from adult cochlea (>P21) revealed a double band for GlyRα3 transcripts, indicated by arrows in FIG. 1 (>P21). GlyRα2 and GlyRα2 transcripts could not be amplified at any of the stages analyzed. Spinal cord cDNA (FIG. 1, sc P21) was used as a positive control for GlyRα1-3, GlyRβ, gephyrin and β-actin.

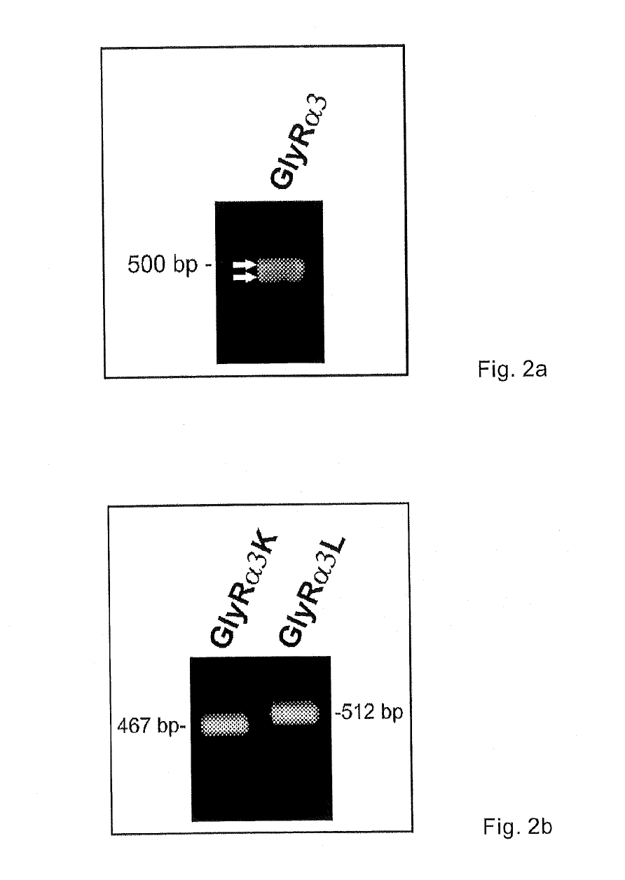

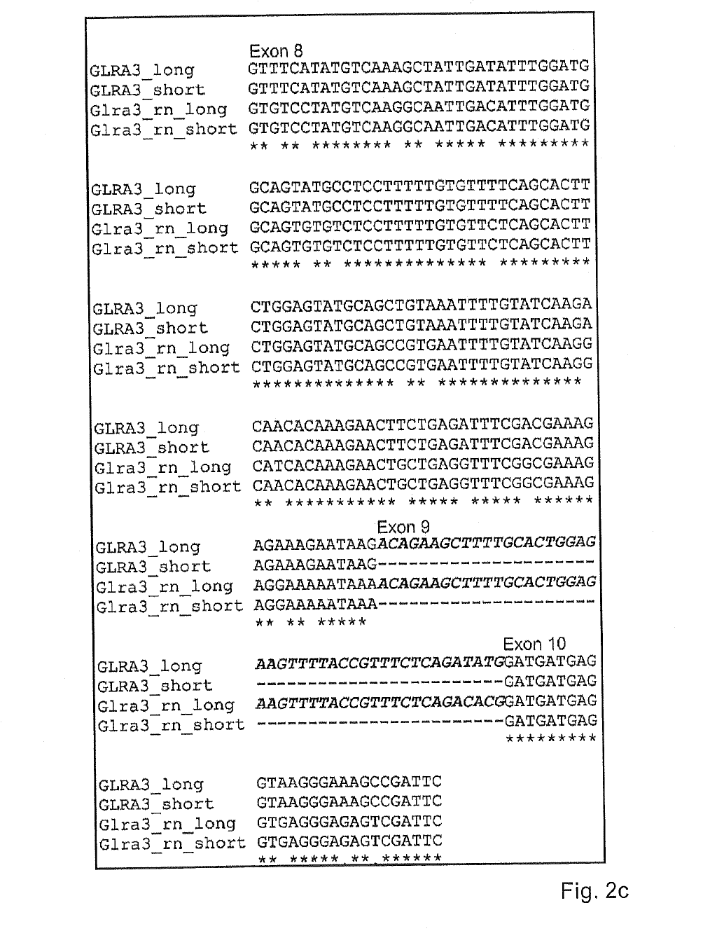

The two PCR products of GlyRα3 double band (FIG. 2a) were cloned into the pCRII-TOPO vector. The sequencing of the insert PCR products revealed two GlyRα3 transcript variants (FIG. 2b). The longer transcript consisting of 512 bp (Glra3_rn_L) showed 99% identity with rat Glra3 cDNA sequence from GeneBank (access number NM_{—}053724). The shorter transcript consisting of 467 bp (Glra_rn_K) carried a 45 bp deletion between nucleotides 1120 and 1164 of the coding sequence (FIG. 2c, in bold italics). The analysis of rat Glra3 and human GLRA3 nucleotide sequences with the Ensemble automatic gene annotation system revealed highly conserved exon-intron borders. The 45 bp deletion in Glra3_rn_K corresponds to missing exon 9 (45 bp) of the human GLRA3_K cDNA sequence (FIG. 2c, exon 9). Thus, the two Glra3 transcripts amplified from rat cochlea exhibited the features of the previously described human GLRA3 short (α3_K) and long (α3_L) splice variants and are therefore referred to as GlyRα3_K and GlyRα3_L in the following text. Sequence data from the GlyRβ and gephyrin PCR fragments confirmed the identity of the amplified transcripts with the corresponding sequences from rat CNS in GeneBank (Glrb: NM_{—}053296; gephyrin: NM_{—}022865, data not shown). In summary, RT-PCR results indicate that GlyRα3, GlyRβ and gephyrin transcripts are expressed in adult rat cochlea from hearing onset (˜P12).

2.2 mRNA Localization of Glycine Receptors and Gephyrin by In Situ Hybridization

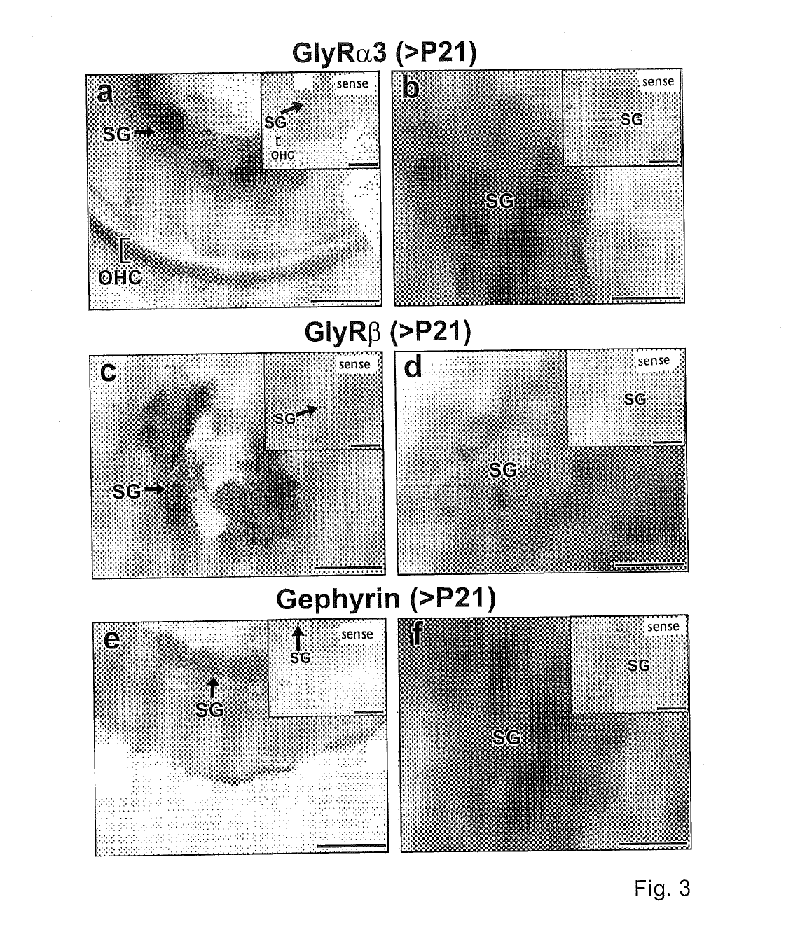

Aiming to gain inside into the localization of GlyR subunit mRNA in the rat cochlea, riboprobes directed against GlyRα3_L, GlyRβ and gephyrin transcripts were produced and in situ hybridization was performed on adult (>P21) rat cochlea. As no sufficient signals were obtained on cryosections after decalcification (data not shown) whole-mount in situ hybridization was employed. In FIG. 3 an overview of a single cochlea turn illustrates signals of GlyRα3 (FIG. 3a), GlyRβ (FIG. 3c), and gephyrin transcripts (FIG. 3e) in spiral ganglion neurons (SG). A higher magnification of the region of the SG indicated a cytoplasmatic localization of GlyRα3 (FIG. 3b), GlyRβ (FIG. 3d) and gephyrin (FIG. 3f) mRNA. The corresponding sense probes (FIG. 3a-f, insets) did not show any signal.

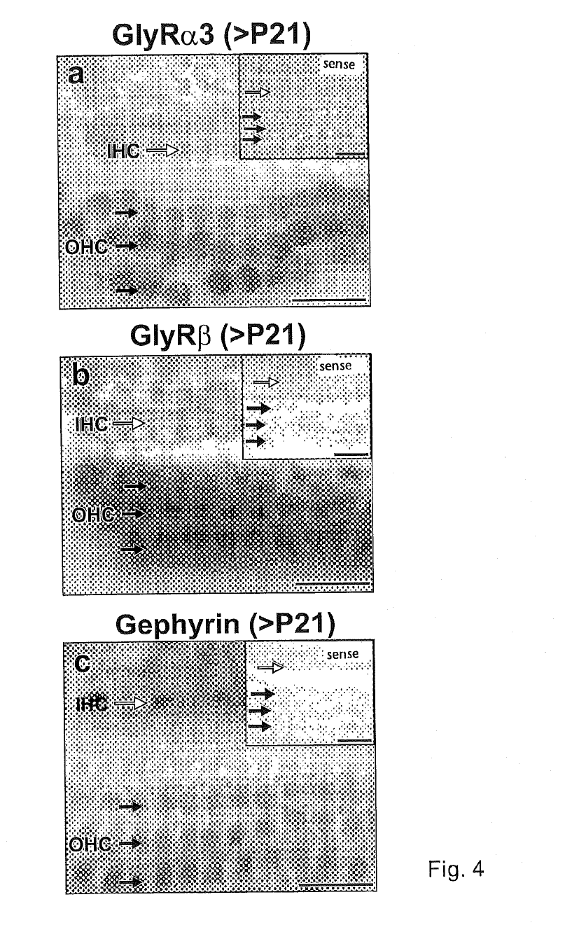

In FIG. 4, the area of hair cells was viewed with higher magnification in the adult rat cochlea. GlyRα3 (FIG. 4a), GlyRβ (FIG. 4b), and gephyrin (FIG. 4c) transcripts were identified in OHCs (filled arrows). While gephyrin and mRNA was detected in addition on the level of IHCs (FIG. 4c, open arrows), no hybridization signal was obtained for GlyRα3 and GlyRβ mRNA (FIG. 4a, b, open arrows) in IHCs. The corresponding sense probes did not produce a signal (insets in FIG. 4a-c). Prior to the onset of hearing, GlyRα3, GlyRβ and gephyrin mRNA was expressed in inner (IHCs) but not in outer hair cells (OHCs) (data not shown).

### 2.3 Protein Detection and Localization of Glycine Receptors and of Gephyrin in the Rat Cochlea

In the next step, it was aimed to visualize GlyRα3 and gephyrin proteins on the hair cell level using a monoclonal mAb4a antibody recognizing all GlyRα subunits, a GlyRα3 specific polyclonal antibody and a monoclonal antibody against the anchoring protein gephyrin.

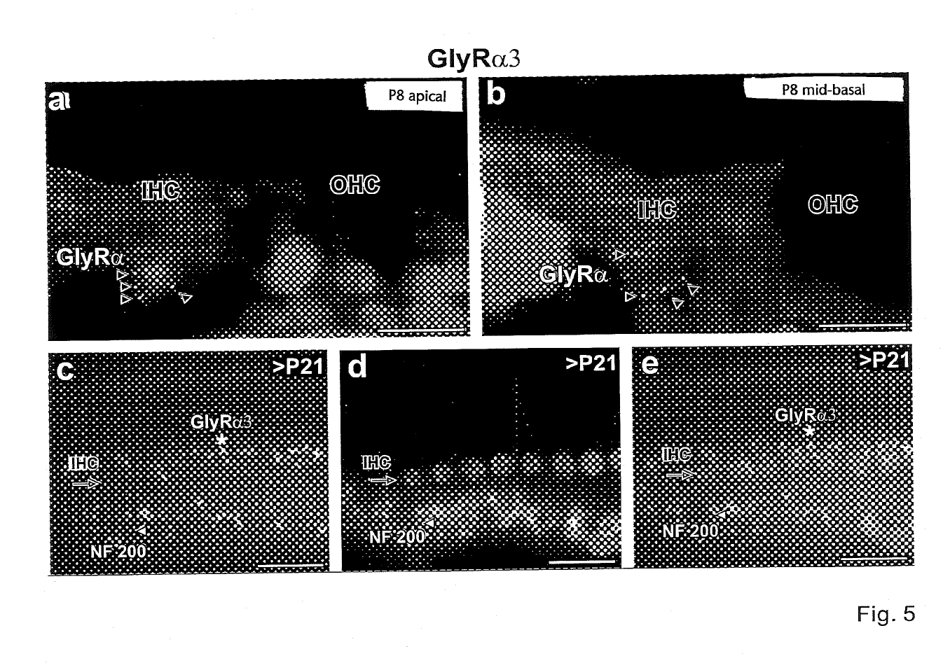

Prior to the onset of hearing, mAb4a, which is directed against a common N-terminal epitope of GlyRα1-4 subunits, detected weak dot-like signals below the inner hair cells, shown for the apical and mid-basal cochlea turn in rat sections at P8 (FIG. 5a, b). At P8, no signal for the GlyRα protein could be observed on the level of the outer hair cells. In contrast to GlyRα, gephyrin polypeptides could not be detected in cryosections, neither in decalcified nor in un-decalcified tissue. In parallel, aiming to circumvent decalcification of the specimens and to improve protein detection, whole-mount immunohistochemistry was used. So far, positive results have been obtained only for the GlyRα3-specific antibody. The dot-like staining pattern was observed for the GlyRα3 protein on the level of IHCs (FIG. 5c, asterisk), close to NF200-immunopositive nerve projections (FIG. 5c, filled arrowhead) shown for rat cochlea >P21. The position of the GlyRα3 staining is illustrated in comparison to the IHC nuclei stained with DAPI (FIG. 5d) or in comparison to the staining of the nuclei and NF200 as a triplet staining (FIG. 5e).

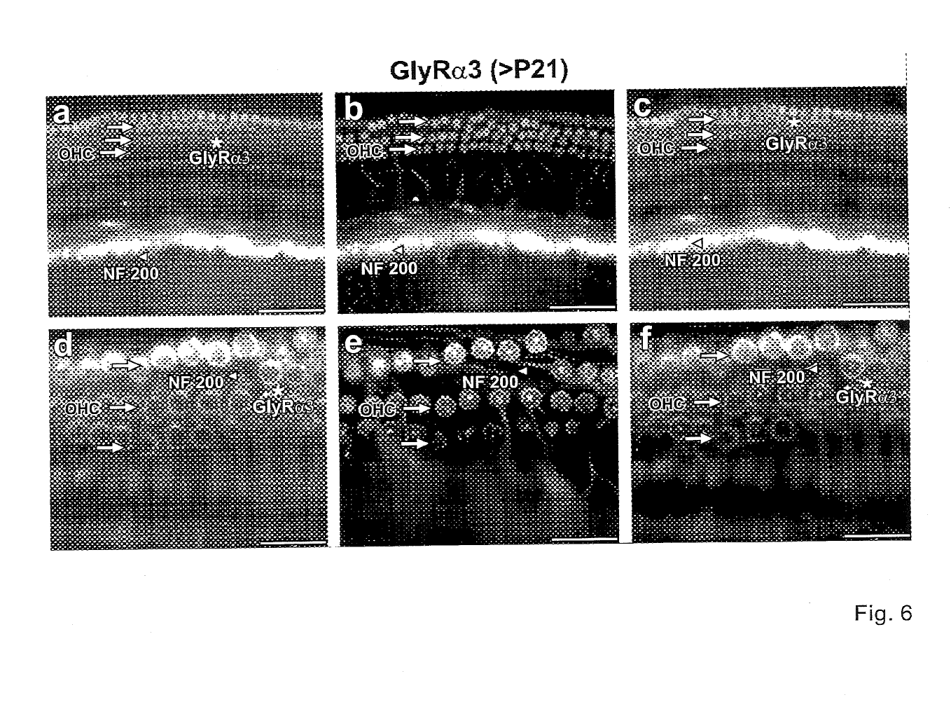

The expression of GlyRα3 protein in OHCs of rats >P21 is depicted in FIG. 6. A signal for the GlyRα3 polypeptide (asterisk) was detected in the three rows of OHCs (FIG. 6a, d, filled arrows). The OHC nuclei were labeled with DAPI (FIG. 6b, c, e, f) and the nerve fibers terminating at the OHCs were stained with an antibody against NF200 (FIG. 6a-f, filled arrowhead). Typical clusters of GlyRα3 protein were located to the cell membrane of OHCs in close proximity to NF200-positive nerve terminals (FIG. 6c, d, f). The omission of primary antibodies did not produce any signal (data not shown).

2.4 The Glycine Receptor Agonist Taurine Inhibits the Upregulation of the Activity of the Auditory Nerve which is Characteristic for Tinnitus

Since an increase of the activity of the auditory nerve after a trauma causally correlates with the induction of (acute) tinnitus in a further experiment it was analyzed whether the local administration of a glycine receptor agonist inhibits the increase of the activity of the auditory nerve and, therefore, is suitable for the treatment of (acute) tinnitus.

By the aid of measurements of compound action potentials (CAP) of the auditory nerves the effect of the glycine receptor agonist taurine (5 μl of a 50 mM solution) and the antagonist strychnine (5 μl of a 10 mM solution), both locally administered, on the activity of the auditory nerve was measured. For this purpose the compound action potentials were activated by different sound stimuli (broadband click stimuli, pure sounds having frequencies of between 4 and 32 kHz, and degrees of loudness from 0 to 100 dB SPL).

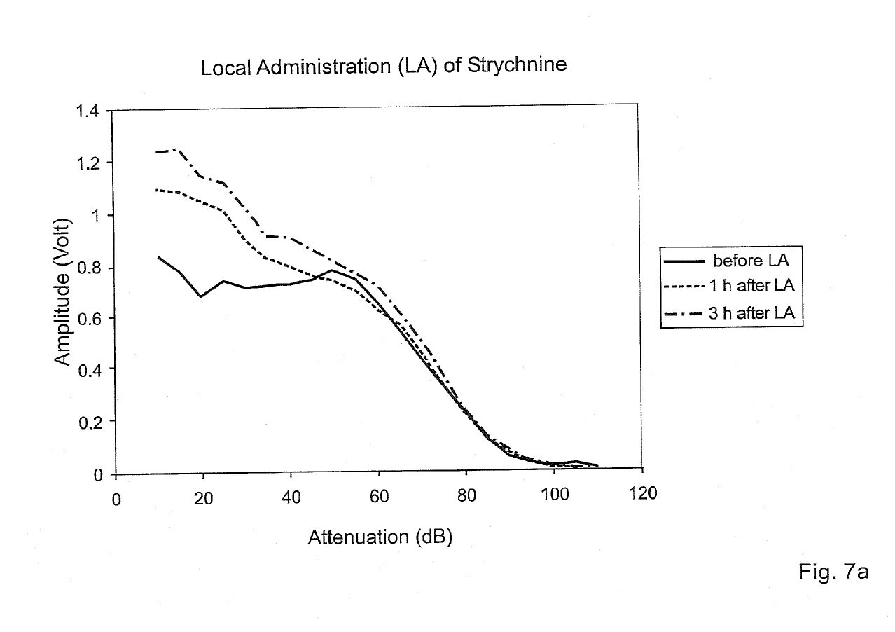

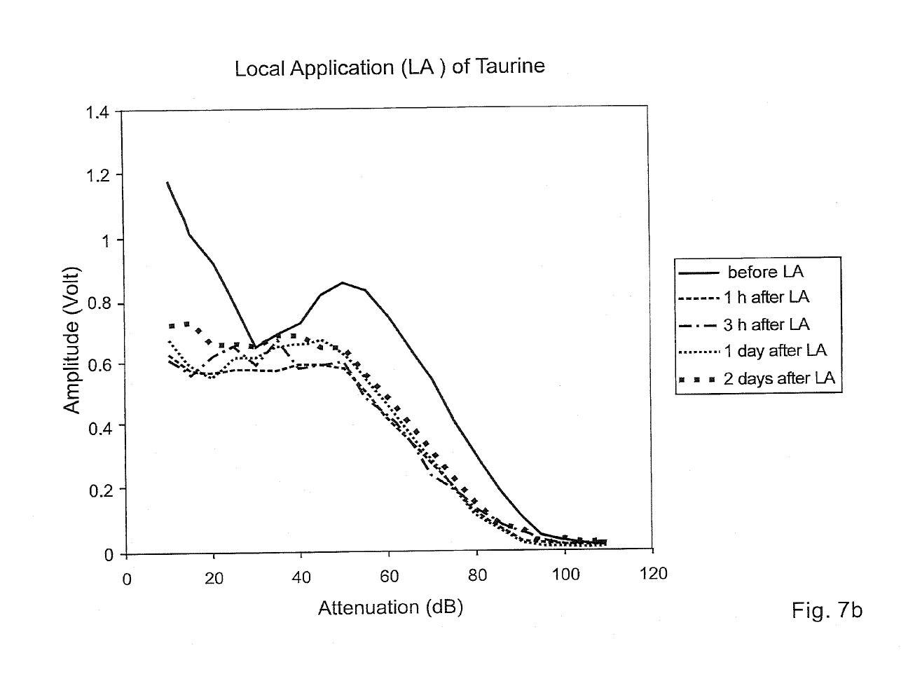

The result is shown in FIG. 7:

At high degrees of loudness (to the left) after a local administration (LA) of strychnine the CAP amplitudes further increase monotonously since the inhibition of the neurons of the auditory nerve which usually starts at high degrees of loudness is absent. This results in an overstimulation at high degrees of loudness; FIG. 7a.

At low to medium degrees of loudness (right) after the local administration (LA) of taurine the CAP amplitudes increase slower, since the glycinergic (inhibitory) neurons which are usually inactive at lower degrees of loudness, were activated by taurine and inhibit the auditory nerve. This results in an inhibition at low and medium degrees of loudness. At high degrees of loudness the glycinergic neurons are activated in any case, therefore in this case an overlapping of the CAP amplitude function can at least be found over a limited range of loudness.

As it can be seen in FIG. 7, within 1-3 hours after the local administration of the glycine receptor antagonist strychnine an increase of the amplitude of the CAP signal with the higher stimulation sound pressure occurs (FIG. 7a, exemplarily shown for measurements at 8 kHz), whereas a reduction of the amplitude of the CAP signals is shown with taurine (FIG. 7b), which was still reduced two days after the local administration at high and low degrees of loudness in relation to the untreated condition (FIG. 7b, “2 days after LA”).

With the method of the CAP measurement the stimulation of the glycinergic efferences by taurines which results in a reduction of the stimulation response at moderate loudness, and the inhibition of the glycinerg efferences which result in increased stimulation responses at high degrees of loudness, can be well described.

The reduction of the activity of the auditory nerve by the local administration of the glycine receptor agonist taurine demonstrates that the group of glycine receptor agonists is suitable to treat or prevent (acute) tinnitus.

### 3. Conclusion

In the present study, glycine receptors and the anchor protein gephyrin was detected in the rat cochlea and their distribution was analyzed by whole-mount in situ hybridization and fluorescence immunohistochemistry. It was demonstrated that by using glycine receptor agonists phantom phenomena, such as the acute tinnitus, can be treated in a causal manner.

### 3.1 Glycine Receptor Isoforms Detected in the Cochlea

The present studies indicate an expression of transcripts of GlyRα3, GlyRβ and gephyrin in the rat cochlea. In contrast, GlyRα1 and GlyRα2 transcripts were not detected at any postnatal nor mature stage analyzed (see FIG. 1).

Among the distinct GlyRα subunit variants, the role of the GlyRα3 subunit has long been elusive. GlyRα transcripts have been detected in the olfactory bulb and cerebellum, the auditory brainstem and the dorsal horn of the spinal cord in adult rodents. Growing evidence for GlyRα3 expression in brain regions associated with sensory processing is indicative of a crucial role of GlyRα3 in sensory integration. This notion is supported by the detection of GlyRα3 in the dorsal horn of the spinal cord, where it has been identified as a key factor in the transmission of pain signals from the periphery to the brain. At the level of the auditory brainstem, glycine receptors play a crucial role in the central sensory processing of acoustic signals, including lateral inhibition and localization of sound sources. The identification of GlyRα3 in the rat cochlea further supports the concept of GlyRα3 as the “sensory” GlyRα subunit variant. To date, it is not understood how the distinct kinetics of GlyRα3 relates to such a role. Recombinant GlyRα3 channels display fast kinetics, yet have a lower affinity for glycine than GlyRα1 channels.

The inventors detected GlyRα3 splice variants corresponding to the human isoforms GlyRα3_K and GlyRα3_L in the rat cochlea (see FIG. 2). So far, alternative splicing of GlyRα3 mRNA has only been described in humans and mice. The short GlyRα3_K isoform lacks the 45 bp-stretch of Exon 9, which corresponds to a loss of 15 amino acids in the channel protein. Recombinant ion channels of the two splice variants exhibit different channel kinetics. The long GlyRα3_L isoform displays a higher affinity for glycine and desensitizes more slowly and to a lesser extent than GlyRα3_K. The screening of cochlea cells for a presumptive differential sub-cellular distribution of GlyRα3 isoforms may be helpful to further elucidate the role of the splice variants. Moreover, electrophysiological recordings from native glycine receptors in isolated hair cells and from recombinant cochlea GlyRα3_K and GlyRα_L channels will help in the characterization of the channel properties of cochlea glycine receptor isoforms and in the understanding of their distinctive role for hearing.

In addition to the ligand-binding GlyRα3 subunit, GlyRβ transcripts were detected in the rat cochlea by RT-PCR and in situ hybridization. To date, it is not known whether native GlyRα3 channels form α3 homopentamers or α3β heteropentamers. Further studies will be required, to elucidate the molecular composition of cochlea glycine receptors. Presumably, gephyrin anchors the cochlea glycine receptors to the cytoskeleton via binding to the β subunit and is crucial for postsynaptic clustering of glycine receptors, as described for the central nerve system and the retina. This is supported by the observation of GlyRα3 protein clusters in immunohistochemical stainings of the immature rat cochlea. The equal distribution of GlyRα3, GlyRβ and gephyrin mRNA in OHCs and SG neurons, which was documented by the inventors (FIG. 3, 4), supports the possibility of α3β heteropentamers in the rat cochlea (see also below).

In the inner hair cells of the mature inner ear, however, only gephyrin mRNA was detected by whole-mount in situ hybridization, whereas no signal for GlyRα3 and GlyRβ mRNA was detected (see FIG. 4c). The expression of gephyrin in regions largely devoid of glyceneric synapses is already described for the CNS and the retina of rodents. There is growing evidence for gephyrin being involved in postsynaptic clustering of GABA_{A} receptors in these regions. While it is necessary to specify the expression of gephyrin transcripts in IHCs in more detail, it is of extreme interest to consider a role of gephyrin as an anchor protein for an IHC-specific ion channel.

### 3.2 Glycine Receptors in the Peripheral Sensory Organs: Retina and Cochlea

In recent years, there has been growing evidence for glycine receptors being involved in the sensory integration in the central nervous system (CNS). Furthermore, the detection and characterization of glycine receptors in the retina gave rise to the hypothesis that glycinergic neurotransmission may also be involved in the peripheral sensory information processing.

In the rodent retina, GlyRα1-4, GlyRβ and gephyrin transcripts where detected at the mRNA and protein level in distinct retinal cell types, indicating a role of glyceneric currents in the processing of visual information in the outer retina. The detection of glycine receptors in the cochlea supports the concept of a modulatory role of glycinergic neurotransmission in peripheral sensory organs.

### 3.3 Glycine Receptors in the Cochlea: Target Molecules of Efferent Innervation

The distribution of GlyR mRNA and protein in the cochlea suggests that the inhibitory glycine receptors and gephyrin are target molecules of the efferent oliviocochlear bundle.

Lateral oliviocochlear (LOC) bundle: The LOC efferent system modulates auditory nerve excitability and balances interaural sensitivity. ACh, GABA, dopamine and CGRP have been identified as transmitters of the LOC system. Prior to the onset of hearing, IHCs are initially contacted by oliviocochlear efferent fibers. In the adult cochlea, these efferent fibers directly contact OHCs and form axosomatic synapses with the afferent dendrites below the IHCs. Therefore, the putative inhibitory receptors of efferent transmitters are presumed to be localized at the time of formation of axosomatic synapses. After the onset of hearing, these receptors are thought to be localized to spiral ganglion neurons and afferent dendrites.

Accordingly, in the adult cochleae the inventors identified transcripts of GlyRα3, GlyRβ, and gephyrin in SG neurons (see FIG. 3) and observed GlyRα3 protein at the IHCs level of neurofilament-positive presumptive afferent fibers (see FIG. 5). Furthermore GlyRα3 protein was localized to the base of IHCs prior to the onset of hearing. The dot-like staining pattern was not only indicative of characteristic GlyR clusters in the cell membrane, it was also reminiscent of the localization of SK2 channel protein in IHCs at the same time point. SK2 proteins are presumed to transmit nicotinic cholinergic receptor-mediated (AChα9, α10) efferent control to IHCs at this early developmental stage.

The inventors have realized that the glycine receptor in the cochlea is of striking clinical and scientific interest. Specifically it has been found that phantom phenomena, such as acute tinnitus and phantom pain, can be treated by the stimulation of glycine receptors in the cochlea by means of glycine receptor agonists.

Medial oliviocochlear (MOC) bundle: It is possible that inhibitoric GlyRs in OHC are involved in efferent signaling of an MOC bundle. After the onset of hearing, nerve fibers of the MOC system contact the basolateral end of OHCs with axosomatic synapses. The MOC bundle works as a sound-evoked feedback loop, which reduces the contribution of OHCs to cochlear amplification and protects the inner ear against acoustic trauma. To date, ACh and GABA have been identified as inhibitory transmitters of the MOC system. The binding of ACh to the α9 nicotinic ACh receptor (nAChR) at the basolateral end of OHC leads to an influence of Ca⁺⁺ ions, which in turn opens the Ca⁺⁺ activated SK2-K⁺ channel. The hyperpolarizing K⁺ efflux creates an inhibitory postsynaptic current (IPSC), which reduces OHC electromotility. In recent years, there has been growing evidence for GABA as a further transmitter of the MOC system. GABA_{A} receptor α and β subunits were detected at the basolateral end of isolated OHCs. Whole-cell recordings of isolated OHCs showed hyperpolarization and elongation of OHCs after application of GABA. These effects were blocked by the GABA_{A} receptor antagonists picrotoxin.

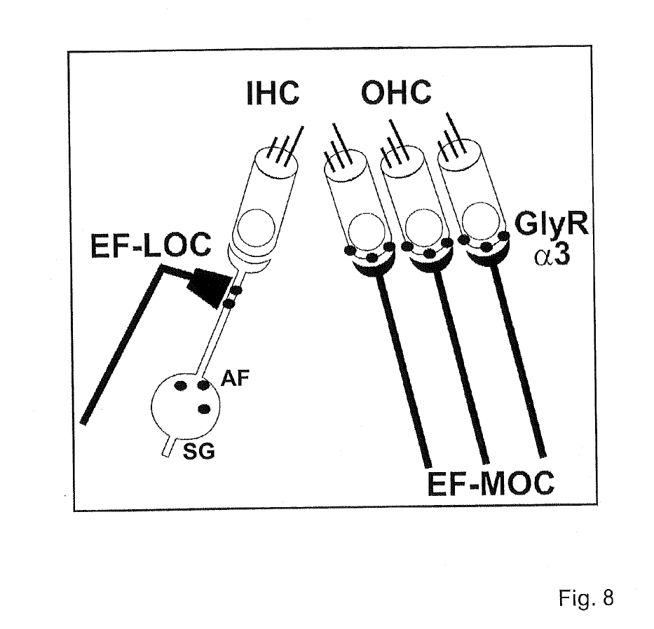

In the studies the inventors could detect GlyRα3, GlyRβ and gephyrin transcripts localized to OHCs by whole-mount in situ hybridization (see FIG. 4). Furthermore, GlyRα3 protein was detected in all three rows of OHCs (see FIG. 6). These findings suggest that inhibitory glycine receptors in OHCs may act as target molecules of the MOC efferent system (FIG. 8, EF-MOC) and contribute to protection of the inner ear against acoustic overexposure. This is supported by the effects of strychnine, the competitive agonist of the inhibitory glycine receptor. One of the prodromal symptoms in strychnine intoxification is hyperacusis. It was so far not known, however, whether this phenomenon is due to central or peripheral disinhibition or both. The observations of the inventors suggest that the inhibitory glycine receptor may contribute to protection against acoustic overexposure. Chronic local administration of strychnine to the inner ear of guinea pigs disrupts efferent activity and results in a permanent threshold shift for high frequencies after acoustic trauma. Currently, these observations are attributed to strychnine acting as an antagonist on the α9-nAChR. Furthermore, recent findings describe a novel AChRα9-mediated strychnine-sensitive component of efferent activity at the OHC level subsequent to high-frequency acoustic stimuli. However, the detection of GlyRs in the inner ear adds a novel clue for the interpretation of strychnine intoxification, suggesting that glycine receptors also contribute to the observed strychnine effects in addition to α9-nAChR in the cochlea.

3.4 Treatment of Phantom Phenomena with Glycine Receptor Agonists and GABA Receptor Agonists

The authors of WO 2006/079476 show a direct correlation of the altered increased expression of BDNF in the periphery of the cochlea and the induction of tinnitus. The increased expression of BDNF in the periphery of the cochlea correlates with a downregulation of the cortical plasticity gene Arg3.1/Arc; cf. Tan et al. (2007), Tinnitus behavior and hearing function correlate with the reciprocal expression patterns of BDNF and Arg3.1/arc in auditory neurons following acoustic trauma, Neuroscience 145(2):715-726.

Both, the upregulation of BDNF in the cochlea as well as the down-regulation of Arg3.1/Arc in the auditory cortex are directly correlatable with induced tinnitus in the animal model; cf. Panford-Walsh et al. (2007), submitted.

Both phenomena of the expression shift of the genes BDNF and Arg3.1/Arc in the cochlea and the auditory cortex, as much as the tinnitus behavior itself can be blocked by the activation of an inhibitory efferent projection which projects axodendritically to the afferent auditory nerves of the inner hair cell; cf. WO 2006/079476.

The inventors were now able to discover a completely new inhibitory transmitter in the inner ear, namely glycine. Specifically, the glycine receptors GlyRα3, GlyRβ, and the anchor protein gephyrin could be detected in the adult cochlea beneath the IHCs and in OHCs.

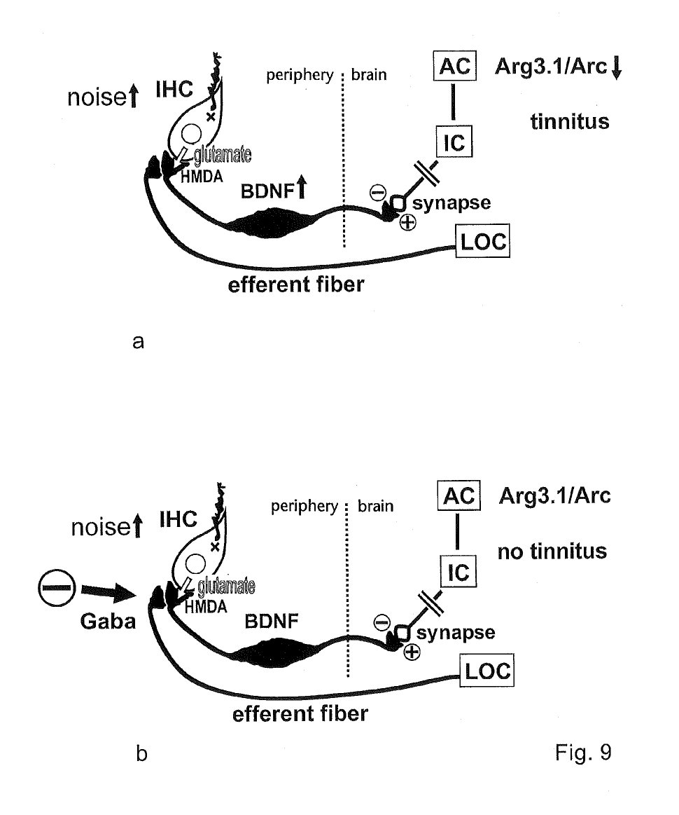

The expression locus of the glycine receptors beneath the inner hair cells (IHCs) shows that, in full analogy to the GABAnergic feedback loop, glycine is secreted by efferences of the medial upper olivio complex (MOC) in the brainstem (FIG. 9b) and, typically, acts inhibitorily on the afferences of the IHCs. The activation of the glycine receptor, belonging to the same receptor type of the “group I” or “cys loop” family like the GABA receptors, results, via the influx of Cl⁻ anions, to the hypopolarization of the neurite. This results in a constant tonic inhibition of the afferent auditory nerve, apparently a significant parameter for the balance of the nerve activity of central auditory projections.

An excitocytosis (too much glutamate) caused by a tinnitus-induced trauma, or a dislocation of the inhibitory “balancing” efferent input, results in the hyperpolarization of the auditory nerve or the increase of the BDNF level in the spiral ganglions, respectively, as already described.

The increase of the BDNF expression is, according to the findings of the inventors, the primary trigger for the induction of tinnitus, which builds a bridge to the pathophysiological alteration of the nerve activity in the central auditory system via the pathological transport of the BDNF protein in the auditory nerve to the first synapse in the brainstem. BDNF acts, depending on the time and duration and amount of the released peptide, on the first postsynapses of the first central auditory switching center in the brain stem, the synapses in the ventral and dorsal nucleus cochlearis. Here the increase of BDNF in the spiral ganglions as observed in tinnitus, can be directly involved in the increase of the activity in the dorsal and ventral nucleus cochlearis and in the inferior colliculus, which, obviously, causes the subsequent reduction of Arg3.1/Arc in the auditory cortex via a detectable increase of inhibitory transmitters, such as GABA. This correlates with a reduction of field potentials in the auditory cortex, an indication for a reduced thalamo-cortical input; cf. FIG. 9a.

A reduction of Arg3.1/Arc in the auditory cortex could directly explain the hyperpolarization or excitocytosis of cortical neurons as already demonstrated in several studies on tinnitus in humans and animals. For a long time the excitocytosis of cortical neurons has been postulated as being the cause for cortical reorganization processes which finally result in phantom perceptions. It was demonstrated in several studies that an upregulation of Arg3.1/Arc proteins in neuronal postsynapses results in a reduced excitatoric postsynaptic potential (EPSP), a downregulation on the other hand results in an increased EPSP. In other words, the overall model of an increase of BDNF in the auditory nerves after the induction of tinnitus can directly explain phenomena which are known for a long time to correlate with tinnitus in humans and animals.

Accordingly, any correction of a pathological increase of BDNF in spiral ganglions should be therapeutically effective. In this model GABA receptor agonists would be effective such as glycine receptor agonists.

Glycine receptor agonists correct in fact, such as GABA receptor agonists, a pathological increase of the BDNF level. As illustrated in FIG. 8B, glycine receptor agonists such as GABA agonists correct the reduction of the cortical expression of Arg3.1/Arc and counteract a pathophysiological reorganization of cortical projections and tinnitus; cf. FIG. 9b.

Summary: An increase of the BDNF levels in the cochlea as found in tinnitus can be met by agonists of an inhibitorily acting input, such as GABA receptor agonists and glycine receptor agonists, directly and locally administered to the auditory nerve. A curative avoidance of an increase of BDNF in the auditory nerve results, in an avoidance of a pathophysiological reorganization of cortical projections which is reflected in the animal model by a reduction of the Arg3.1/Arc expression in the cortical neurons with increased EPSP.

What is claimed is:

1. Method for the treatment of the phantom phenomena of acute tinnitus and/or phantom pain in a human being, comprising the following steps:
(a) Providing a medicament,
(b) administering the medicament to the human being, and, if applicable,
(c) repeating the steps of (a) and (b),
wherein the medicament comprises a glycine receptor agonist.

2. Method of claim 1, wherein as a glycine receptor agonist a substance is provided which is selected from the group consisting of: D-alanine, L-alanine, L-serine, taurine, cannabinoid, tropine, nortropine and derivatives thereof.

3. Method of claim 2, wherein the cannabinoid is selected from the group consisting of anandamide, arachidonylglycerol, tetrahydrocannabinol, WIN 55,212-2.

4. Method of claim 1, wherein in step (b) the substance is locally administered at or into the ear or the site of the amputation.

5. Method of claim 4, wherein the local administration into the ear in step (b) is realized via the round window membrane.

6. Method of claim 1, wherein the medicament comprises an additional substance effective against phantom phenomena, selected from the group consisting of GABA receptor agonists, in particular benzodiazepines and substances related thereto, baclofen, gamma vinyl GABA, gamma acetylene GABA, progabid, muscimol, iboten, sodium valproate and tetrahydroisoxazolopyrdine (THIP), MAP kinase inhibitors, in particular U 0126 or PD 98058, Cam kinase inhibitors, L-type Ca⁺⁺ channel antagonists, in particular nicardipin or nifedipin or isradipin, CREP antagonists, glutamate antagonists, trkB antagonists.

7. Method for the production of a medicament for the treatment of the phantom phenomena of acute tinnitus and/or phantom pain in a human or animal being, comprising the following steps:
(a) Providing a glycine receptor agonist, and
(b) formulating the glycine receptor agonist into a pharmaceutically acceptable carrier.
